(19) United States
(12) Plant Patent Application Publication
(10) Pub. No.: US 2026/0363155 P1
(43) Pub. Date: Nov. 12, 2026

(54) Hydrangea Plant Named 'Darbi'

(50) ;

(71) Applicant: Jean-Paul DAVASSE, Loire Authion (FR)

(72) Inventor: Jean-Paul DAVASSE, Loire Authion (FR)

(21) Appl. No.: 19/673,603

(22) Filed: May 11, 2026

(30) Foreign Application Priority Data
May 12, 2025 (QZ) 20250986

(51) Int. Cl. A01H 6/48; A01H 5/02

(52) U.S. Cl. PLT/250

(57) ABSTRACT

A new cultivar of Hydrangea macrophylla x serrata plant named ‘Darbi’ that is characterized by its dark colored foliage from bud opening into the fall and remains dark through the summer, its round ball-shaped inflorescences, its reddish colored veins on young leaves, green colored stems that are striated with black, its semi-erect plant habit, its sterile flower sepals that are white and red in color, its inflorescences that fade darker in color at the end of summer, its blooms that are not affected by spring frost as it has terminal, lateral, and basal buds.

### CROSS REFERENCE TO A RELATED APPLICATIONS

This application claim priority to a European plant breeders' rights application filed on May 12, 2025, application No. 2025/0986 and is also related to a Canadian plant breeders' rights application filed Feb. 6, 2026 application No. 26-12299. There have been no offers for sale anywhere in the world prior to the effective filing date of this Application and no accessibility to one of ordinary skill in the art could have been derived from the printed plant breeder's rights documents.

Botanical classification: Hydrangea macrophylla x serrata.

Varietal denomination:

‘Darbi’.

### BACKGROUND OF THE INVENTION

The present invention relates to a new and distinct cultivar of Hydrangea macrophylla x serrata and will be referred to hereafter by its cultivar name, ‘Darbi’. ‘Darbi’ represents a new mophead type Hydrangea, a deciduous shrub grown for use as a landscape plant.

‘Darbi’ was derived from an ongoing breeding program conducted by the Inventor in Sainte Gemmes sur Loire, France. A goal of the breeding program is to develop new cultivars of Hydrangea macrophylla x serrata that have dark green foliage and ball formed inflorescences.

The Inventor made a controlled crosses in June of 2018 in Sainte Gemmes sur Loire, France between Hydrangea macrophylla x serrata ‘JPD01’ (U.S. Plant Pat. No. 33,412) as the female parent and an unnamed and unpatented proprietary plants from the Inventor's breeding program as the male parents. The female parent was pollinated by the pollen from several male parents, and the seeds were pooled and therefore the exact male parent is unknown. ‘Darbi’ was selected in July of 2019 as a single unique plant amongst the resulting seedlings.

Asexual propagation of the new cultivar was first accomplished under the direction of the Inventor by softwood stem cuttings in May of 2020 in Sainte Gemmes sur Loire, France. Asexual propagation by softwood stem cuttings has determined that the characteristics of the new cultivar are stable and are reproduced true to type in successive generations.

### SUMMARY OF THE INVENTION

The following traits have been repeatedly observed and represent the characteristics of the new cultivar. These attributes in combination distinguish ‘Darbi’ as a unique cultivar of Hydrangea. p1 1. ‘Darbi’ exhibits dark colored foliage from bud opening into the fall and remains dark through the summer.
2. ‘Darbi’ exhibits round ball-shaped inflorescences.
3. ‘Darbi’ exhibits reddish colored veins on young leaves.
4. ‘Darbi’ exhibits green colored stems that are striated with black.
5. ‘Darbi’ exhibits a semi-erect plant habit.
6. ‘Darbi’ exhibits sterile sepals that are white and red in color.
7. ‘Darbi’ exhibits blooms that are not affected by spring frost as it has terminal, lateral, and basal buds.

The female parent of ‘Darbi’ differs from ‘Darbi’ in having sepals that are pinkish red in color, inflorescences that are lacecap in form, a more compact plant habit, and stems that are tinged with a red color. ‘Darbi’ can be most closely compared to the Hydrangea macrophylla cultivars ‘Bailmacseven’ (U.S. Plant Pat. No. 34,544) and ‘Ripple’ (U.S. Plant Pat. No. 15,787). ‘Bailmacseven’ is similar to ‘Darbi’ in having dark green colored foliage that retains color throughout the summer and mophead-shaped inflorescences with sterile flower sepals with a pink-red edge. ‘Bailmacseven’ differs from ‘Darbi’ in having an upright mounded habit, green colored veins on young leaves, red-green stems, and elliptical shaped leaves. ‘Ripple’ is similar to ‘Darbi’ in having mophead shaped inflorescences.

‘Ripple’ differs from ‘Darbi’ in having leaves that turn green in the summer when temperatures are hot and foliage that is sensitive to mildew, and in being sensitive to spring frost.

### BRIEF DESCRIPTION OF THE DRAWINGS

The accompanying color photographs illustrates the overall appearance and distinct characteristics of the new Hydrangea. The photographs were taken of a 4-year-old plant as grown in a 5-liter container in Sainte Gemmes sur Loire, France.

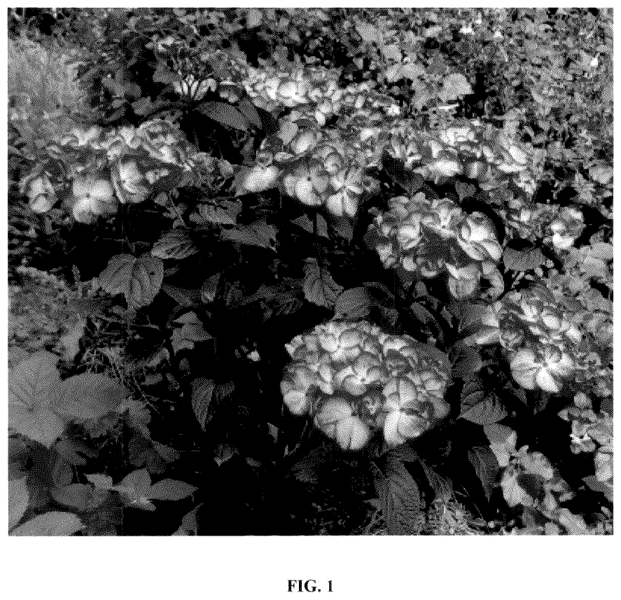

The photograph in FIG. 1 provides a side view of the plant habit of ‘Darbi’ in bloom.

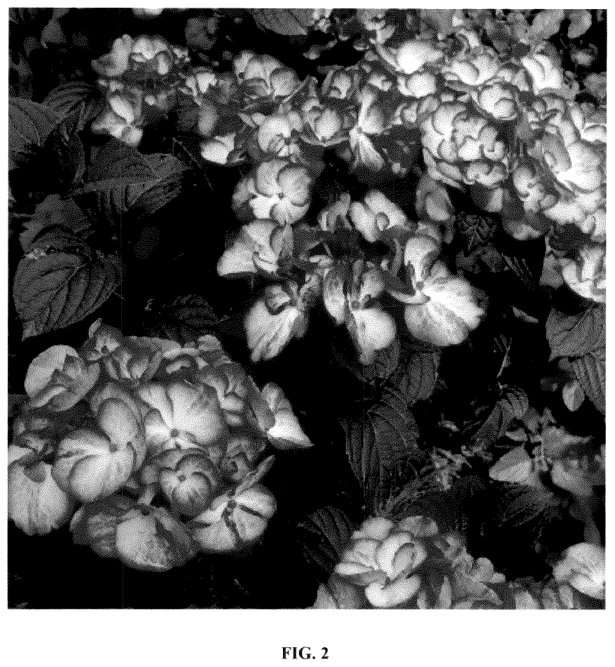

The photograph in FIG. 2 provides a close-up view of the inflorescences of ‘Darbi’.

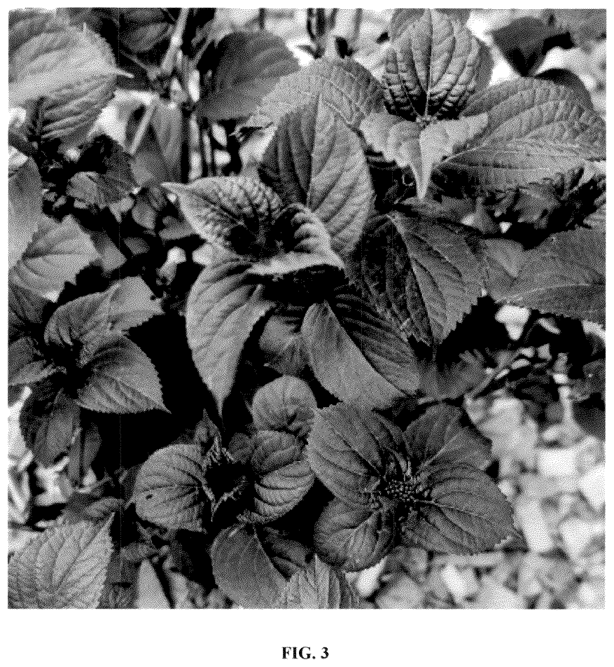

The photograph in FIG. 3 provides a close-up view of the foliage of ‘Darbi’.

The colors in the photographs may differ slightly from the color values cited in the detailed botanical description, which accurately describe the colors of the new Hydrangea.

### BOTANICAL DESCRIPTION OF THE PLANT

The following is a detailed description of 2-year-old plants of ‘Darbi’ as grown in 2-gallon containers inside a double poly house in Abbotsford, BC, Canada. The phenotype of the new cultivar may vary with variations in environmental, climatic, and cultural conditions, as it has not been tested under all possible environmental conditions. The color determination is in accordance with The 2015 Colour Chart of The Royal Horticultural Society, London, England, except where general color terms of ordinary dictionary significance are used.
General description:
   Blooming period.—From the second week in June until September in Angers, France.
   Plant type.—Deciduous shrub, mophead type Hydrangea.
   Plant habit.—Semi-upright.
   Height and spread.—Average of 1.4 m in height and 1.2 m in spread as grown in the landscape.
   Hardiness.—At least in U.S.D.A. Zones 4 to 9.
   Diseases and pests.—No resistance or susceptibility to diseases or pests has been observed.
   Root description.—Fine and fibrous, 161A.
   Propagation.—Softwood stem cuttings.
   Root development.—Time required for root initiation is 3 weeks at 18 °C night temperatures, time required to produce a young plant from rooted cutting is an average of 3 months.
Branch description:
   Branch shape.—Rounded.
   Branch strength.—Strong.
   Branch color.—Young; 144A, flushed with N186A and 202A, mature and older branches; N186A and 202A.
   Branch size.—An average of 30 cm in length, 4 mm in diameter.
   Branch surface.—Younger and mature branches are glabrous and glossy, moderately covered with lenticels; oblong in shape, 1 mm in length, N186A and 202A in color, older branches bark-like.
   Branch aspect.—Held in multiple angles, upright to slightly outward.
   Internode length.—Average of 4.5 cm.
   Branching.—1 main branch, 8 lateral branches per plant.
Foliage description:
   Leaf shape.—Ovate.
   Leaf arrangement.—Opposite.
   Leaf division.—Simple.
   Leaf base.—Cuneate.
   Leaf apex.—Cuspidate.
   Leaf margins.—Dentate-serrate.
   Leaf venation.—Pinnate, upper surface color; 185A, lower surface color; 148A, flushed with 185A.
   Leaf size.—Average of 7 cm in length and 5 cm in width.
   Leaf attachment.—Petiolate.
   Leaf surface.—Both surfaces smooth and velvety.
   Leaf color.—Young upper surface; 144A, N186A, 187A, young lower surface; 144A, lightly flushed with 200A, mature upper surface N186A, mature lower surface; a blend of N199A and 197A, flushed with 187A, older leaves upper surface; 185A, older leaves lower surface; 176B.
   Petioles.—An average of 1 cm in length and 3 mm in diameter, surfaces are glossy and smooth, color; 183A, flushed with N186A.
Inflorescence description:
   Inflorescence type.—Flattened, compound corymb of single sterile and fertile flowers.
   Lastingness of inflorescence.—Sterile flowers; persistent, lasting about 6 weeks, fertile flowers; self-cleaning, lasting about 1 week.
   Inflorescence number.—One per branch.
   Inflorescence size.—Average of 4 cm in height and 10 cm in diameter.
   Flower number.—An average of 25 sterile flowers and 6 fertile flowers per inflorescence.
   Flower fragrance.—None.
   Flower aspect.—Sterile flowers; upright to outward and slightly drooping, fertile flowers; upright to outward.
   Flower size.—Sterile flowers; rotate in shape, average of 3.2 cm in diameter and 1.5 cm in depth, fertile flowers; rotate, average of 5 mm in diameter and 4 mm in depth.
   Peduncles.—Average of 1.9 cm in length and 2 mm in width, moderately strong, surface is glabrous and glossy, color; NN155D, flushed with 187A.
   Pedicels.—Sterile Flowers; average of 1 cm in length and 1.5 mm in diameter, moderately strong, NN155D in color, glabrous and glossy surface, fertile flowers; average of 4 mm in length and 1 mm in diameter, moderately strong, NN155D in color, glabrous and glossy surface.
   Petals.—Sterile flowers; petal spot (flower bud); 2 mm in depth and 1.5 mm in diameter, round in shape, color; 63A, N45A, and 155A, petals do not open, fertile flowers; petal spot (flower bud); 1.2 mm in depth, 1.5 mm in diameter, round in shape, color; 63A, N45A, and 155A, when petals open; average of 5, rotate, concave in shape, acute apex, cuneate base, entire margins, 3 mm in length and 2 mm in width, both surfaces are glabrous, satiny, and matte, color: both surfaces when opening and fully open 63A, N45A, and 155A, petals drop after maturity.
   Sepals.—Sterile flowers; showy, 4 to 5, rotate in arrangement, moderately overlapping margins, both surfaces smooth and satiny, depressed (very shallow) round in shape, entire margins, slightly undulate, emarginate apex, cuneate base, an average of 1.5 cm in length and width, color: upper and lower surface when opening; center 155A and 150D, flushed with 61A, edges N45A, upper and lower surface when fully open; center 155A, flushed with 63A, edges N45A, fading sepals upper and lower surface; N155D, edges lightly flushed with 63D, outer edges 63A, fertile flowers; 5, in a star-shaped arrangement, round to oval in shape, round apex, flat base, entire margins, average of 1.5 mm in length and 1 mm in width, both surfaces smooth and satiny, both surfaces 71A in color.
Reproductive organs: (Present on fertile flowers).
   Androecium.—Sterile flowers; none present, fertile flowers; stamens; average of 8, filaments; average of 3 mm in length and NN155D in color, anthers; round in shape, 0.5 mm in diameter, NN155D in color, pollen; none observed.
   Gynoecium.—Sterile flowers; none present, fertile flowers; pistils; average of 3, 2 mm in length, stigma; club-shaped and NN155D in color, style; 0.5 mm in length and NN155D in color, ovary; 145B in color.
   Fruit and seed.—No seeds or fruit observed to date.

It is claimed:

1: A new and distinct cultivar of Hydrangea plant named ‘Darbi’ substantially as herein illustrated and described.
